SEQUENCE LISTING (15 sequences)

SEQ ID NO: 1 - protein, 43 aa, Glycine max
  Lunasin
SKWQHQQDSCRKQLQGVNLTPCEKHIMEKIQGRGDDDDDDDDD

SEQ ID NO: 2 - protein, 40 aa, Glycine max
  Lunasin-GRG
SKWQHQQDSCRKQLQGVNLTPCEKHIMEKIQDDDDDDDDD

SEQ ID NO: 3 - protein, 21 aa, Glycine max
  trLunasin
EKHIMEKIQGRGDDDDDDDDD

SEQ ID NO: 4 - protein, 10 aa, Glycine max
  trLunasin-del
EKHIMEKIQG

SEQ ID NO: 5 - protein, 25 aa, Glycine max
  NLS-trLunasin
LEEKQKKKMEKEQGRGDDDDDDDDD

SEQ ID NO: 6 - protein, 11 aa, Glycine max
  Lunasin
CEKHIMEKIQG

SEQ ID NO: 7 - protein, 12 aa, Glycine max
  HuHP1(p25)
EEEEYVVEKVLD

SEQ ID NO: 8 - protein, 12 aa, Glycine max
  DmPc
VDLVYAAEKIIQ

SEQ ID NO: 9 - protein, 12 aa, Glycine max
  Hu HP1B
FERGLEPEKIIG

SEQ ID NO: 10 - protein, 12 aa, Glycine max
  SpSw16A
EEDEYVVEKVLK

SEQ ID NO: 11 - protein, 12 aa, Glycine max
  PcHET2A
VEEEFIVEKILD

SEQ ID NO: 12 - protein, 12 aa, Glycine max
  DvHP1A
EEEEYAVEKILD

SEQ ID NO: 13 - protein, 12 aa, Glycine max
  MoMOD1A
EEEEYVVEKVLD

SEQ ID NO: 14 - protein, 12 aa, Glycine max
  SmPAT26
GEDEFQVEKILK

SEQ ID NO: 15 - protein, 12 aa, Glycine max
  DmHP1A
EEEEYAVEKIID

(19) United States
(12) Patent Application Publication
(10) Pub. No.: US 2003/0027765 A1
(43) Pub. Date: Feb. 6, 2003

(54) Therapeutic peptides having a motif that binds specifically to non-acetylated H3 and H4 histones for cancer therapy

(76) Inventor: Alfredo F. Galvez, Davis, CA (US)

Correspondence Address: Howard M. Peters, PETERS, VERNY, JONES & SCHMITT LLP, Suite 6, 385 Sherman Avenue, Palo Alto, CA, 94306, US

(21) Appl. No.: 10/252,256

(22) Filed: Sep. 23, 2002

Related U.S. Application Data
(63) Continuation-in-part of application No. 09/534,705, filed on Mar. 24, 2000.
(63) Continuation-in-part of application No. PCT/US01/09453, filed on Mar. 23, 2001.

(51) Int. Cl. A61K 38/17

(52) U.S. Cl. 514/12

(57) ABSTRACT

The present invention describes a composition of matter comprising of a conserved structural motif that allows the targeting and binding of a chromatin binding protein to non-acetylated histone H3 and H4 and prevents their acetylation. This invention is responsible for the anti-carcinogenic property of a chromatin binding peptide isolated from soybean seed. This structural motif is found in a highly conserved manner in other chromatin-binding proteins from different species. Modifications to this structural motif such as fusions to other proteins with functional motifs and amino acid substitutions have potential therapeutic applications and can be developed as an in vivo gene silencing technology for biological and medical research. In particular, active fragments of the lunasin peptide and active analogs of the lunasin peptide are useful in this invention. Pharmaceutical compositions useful in retarding or stopping or reducing various types of cancers are described.

### RELATED APPLICATIONS

This application is a continuation-in-part of U.S. Ser. No. 09/534,705, filed Mar. 24, 2000, which is incorporated herein by reference in its entirety.

### BACKGROUND OF THE INVENTION

1. Field of the Invention

This invention relates to lunasin, its fragments, analogs and the like which have a defined helical moiety which comprises a structurally conserved helical motif, a stretch of polyacidic amino acids (either aspartic or glutamic acid) and an Arg-Gly-Asp (RGD) for lunasin targeting and binding to non-acetylated N-terminal tails of H3 and H4 histones, making them unavailable for acetylation, and for cell membrane adherence and internalization. The substances are useful in a variety of disease therapy including reduction/repression of existing cancer or prevention of cancer initiation.

2. Description of Related Art

Lunasin the Small Subunit of a Soybean 2S Albumin, Colocalizes with Endoreduplicated Genomic has DNA in Storage Cells of Developing Seed.

The lunasin peptide with its unique poly-apartic acid carboxyl end and was proposed to have an important biological function when it was isolated and sequenced but not cloned from soybean seeds by a Japanese group 13 years ago (Odani et al., 1987 J Biol Chem, vol 262:10502). However, only upon the isolation and cloning of the Gm2S-1 cDNA could a putative biological role for lunasin be inferred. The Gm2S-1 cDNA encodes lunasin as a 43 amino acid small subunit component of a post-translationally processed 2S albumin (Galvez et al., 1997 Plant Physiol, vol. 114:1567). Gm2S-1 expression occurs only in the cotyledon and coincides with the initiation of mitotic arrest and DNA endoreduplication in developing soybean seed (Galvez et al., 1997). DNA endoreduplication is a unique cell cycle of G1 and S phases without cell division that occurs only in terminally differentiated storage parenchyma cells (Goldberg et al,1994 Science, vol. 266:605). In situ hybridization experiments using a lunasin antisense RNA probe and immunolocalization using a polyclonal antibody raised against the carboxyl end of lunasin, showed lunasin expression in storage parenchyma cells undergoing DNA endoreduplication and cell expansion but not in actively dividing cells of the cotyledon (FIGS. 1A, 1B, 1C, 1D and 1E.).

The temporal and spatial expression of lunasin in developing seeds suggest a biological role of lunasin as an effector molecule that inhibits cell division and allows DNA endoreduplication and cell expansion to occur in storage parenchyma cells during seed development. Its colocalization with endoreduplicated genomic DNA suggests a potential role as a repressor of gene expression in newly replicated genomic DNA. Despite the presence of multiple copies of the genome, the level of gene expression in storage parenchyma cells corresponds to a single copy of the genome. By binding to hypoacetylated chromatin associated with newly replicated DNA, lunasin is thought to silence expression of genes in the reduplicated genome by forming repressed chromatin structures. In addition, lunasin binding to hypoacetylated chromatin could inhibit mitotic condensation of the chromosomes and prevent microtubule nucleation, leading to the failure of cell division in expanding storage parenchyma cells. In support of this hypothesis, studies have shown that the phosphorylation of serine 10 in the amino terminal tail of histone H3 is required for the proper segregation and condensation of chromosomes during mitosis (Wei et al., 1999 Cell, vol. 97:99). Lunasin as described below has preferential binding affinity to the non-acetylated amino terminal tails of histone H3 and H4. By making the serine 10 unavailable for phosphorylation as a result of lunasin binding to the H3 amino terminal tail, lunasin can prevent condensation of chromosomes and consequently inhibit cell division.Constitutive expression of lunasin in mammalian cells

Disrupt Centromere Assembly and Mitosis.

The temporal and spatial expression oflunasin coincide with the initiation of mitotic arrest and DNA endoreduplication in developing soybean cotyledon. This information, together with the observation that lunasin expression caused aberrant cell division in bacteria (Galvez and de Lumen, 1999 Nature Biotechnology, vol. 17:495), led to the hypothesis that lunasin should also disrupt eukaryotic cell division. To test this hypothesis, a chimeric gene encoding the lunasin peptide tagged with green fluorescent protein (GFP) was constructed. The transient transfection of the GFP-lunasin construct arrested cell division, caused abnormal spindle fiber elongation, chromosomal fragmentation and cell lysis in murine embryo fibroblast, murine hepatoma, and human breast cancer cells (Galvez and de Lumen, 1999). Transfection of a control construct with a deleted poly-aspartyl end abolished lunasin's antimitotic effect.

The mechanism of action of other antimitotic agents such as vinblastine, colchicine, nocodazole and taxol involves the disruption of mitotic spindle dynamics during mitosis. Unlike these compounds, lunasin disrupts mitosis in mammalian cells by binding to chromatin and preventing the formation of the kinetochore complex in the centromere. This is likely brought about by the binding of the negatively charged lunasin to the highly basic histones found within the nucleosomes of condensed chromosomes, particularly to regions that contain more positively charged, hypo-acetylated chromatin such as found in telomeres and centromeres. The displacement by lunasin of the kinetochore proteins normally bound to the centromere leads to the failure of spindle fiber attachment, and eventually to mitotic arrest and cell death. The observations of lunasin adhering to the fragmenting chromosomes after cell lysis, the asymmetric distribution of metaphase chromosomes, the elongated spindle fibers, and the unattached kinetochores observed in lunasin-transfected cells are consistent with this proposed model for the mechanism of action of lunasin (Galvez and de Lumen, 1999).

Lunasin Peptide Adheres to Mammalian Cell Membrane Gets Internalized and Binds to Regions of Hypoacetylated Chromatin (i.e. Telomeres)

Lunasin contains the cell adhesion motif RGD (arg-gly-asp). Synthetic and recombinant peptides containing the RGD motif derived from sequences of extracellular matrix proteins like fibronectin, have been shown to bind to specific membrane integrins in mammalian cells (E. Ruoslahti, M. D. Piersbacher. Cell, vol. 44, 517 (1986); S. K. Akiyama, K. Olden, K. M. Yamada. Cancer Metastasis Rev., vol. 14, 173 (1995)). To determine whether lunasin has a functional RGD motif, a cell adhesion assay using synthetic lunasin peptides and mice embryo fibroblast cells (C3H 10T½) was conducted (L. M. De Luca, et al., Methods of Enzymol, vol. 190:81-91 (1990)). The lunasin peptide adhered to C3H cells in a dose-dependent manner and that the deletion of the RGD tripeptide from lunasin (Lunasin-GRG) prevented cell adhesion (FIG. 2). When applied exogenously to the growth media, lunasin was not only adhering to the cell membrane but became internalized as well, preferentially binding to the telomeres of chromosomes during metaphase (FIGS. 3A, 3B, 3C, 3D, 3E, and 3F). However, unlike the constitutive expression of lunasin gene in transfected cells that disrupts kineto chore formation (Galvez and de Lumen, 1999 ), internalized lunasin did not affect kinetochore assembly. Immunostaining experiments showed the normal kinetochore location of the cell cycle checkpoint protein, MAD (Y. Li; R. Benezra, Science, vol. 274,246 (1996); R. H. Chen, J. C. Waters, E. D. Salmon, A. W. Murray, Science, vol. 274, 242 (1996)), in the centromere of metaphase chromosomes. As a result, the exogenous application of lunasin did not affect cell division and proliferation of murine embryo fibroblast cells. Immunostaining using the lunasin polyclonal antibody also showed that internalized lunasin was initially found in the cytoplasm and then eventually bound to hypoacetylated regions of the chromosome, such as those in the telomeres, upon nuclear membrane breakdown at prometaphase (FIGS. 3A, 3B, 3C, 3D, 3E, and 3F.). However, at this stage of mitosis, kinetochore assembly and spindle fiber attachment to centromeres had already transpired. This explains the non-disruptive effect of exogenously applied lunasin on cell division as compared to the antimitotic effect observed when lunasin is constitutively expressed in lunasin-transfected mammalian cells (Galvez and de Lumen, 1999).

A U.S. patent if interest is U.S. Pat. No. 6,107,287 issued Aug. 23, 2000.

All articles, references, standards, patents, patent applications and the like cited in this application are hereby incorporated herein by reference in their entirety.

With regard to the above background description, there exists a significant need to provide-a method and pharmaceutical composition to inhibit or retard various cancers from initializing and or reducing existing cancers for shrinking particularly in a human being. The present invention provide such a method and pharmaceutical composition.

### SUMMARY OF THE INVENTION

The present invention relates to a method of cancer treatment or prevention, which method involves:

A. Administering to a mammalian subject having tumor cells in need of therapy or a mammalian subject at risk to carcinogen-mediated cancer formation an effective amount of an isolated and purified therapeutic agent selected from the group consisting of lunasin peptide, an active fragment of lunasin peptide, an active lunasin peptide analog, and combinations thereof which lunasin moiey has a helical portion which comprises the structural motif (ED)NNXXXEK(IV), where E is glutamic acid, D is aspartic acid, K is lysine, I is isoleucine, V is valine, X is conserved hydrophobic amino acids and N is any amino acid, a sequence of at least 5 up to 15 poly-acidic amino acids (glutamic or aspartic acids), and an Arg-Gly-Asp (RGD) motif which is useful for targeting and binding to non-acetylated N-terminal tails of H4 and H3 histones and for functional adhesion of lunasin moiety to the outer cell membrane;

B. Causing the lunasin peptide, the active fragment of lunasin peptide, the active lunasin peptide analog or combinations thereof to contact and to adhere to the functional cell membrane;

C. Causing the lunasin peptide, the active fragment of lunasin peptide, the active lunasin peptide analog or combinations thereof to contact and to become internalized within the functioning cell;

D. Causing the lunasin peptide, the active fragment of lunasin peptide, the active lunasin peptide analog or combinations thereof to preferentially bind to the deacylated N-terminal portions of histone H3 and H4, causing these histones to be unavailable for further acylation in regions of the chromosomes of the cell and which are enriched with hypoacylated repressed chromatin;

E. Inducing apoptosis of the cell by repression of carcinogen and oncogene-mediated gene expression within the cell; and

F. Resulting in significantly reduced or termination of cancer activity of existing tumor cells or the prevention of significant tumor cell initiation.

The method wherein the mammal is a human being.

The method wherein the method is one of treating an already existing cancer.

The method wherein the method is one of preventing or repressing the induction of cancer.

The method wherein the therapeutic agent comprises lunasin peptide.

The method wherein the therapeutic agent comprises an active fragment of lunasin peptide.

The method wherein the therapeutic agent comprises an active analog of lunasin peptide.

The method wherein the therapeutic agent is administered orally, topically, intranasally, intramuscularly, subcutaneously, intraperioneally, buccally or combinations of these methods.

The method wherein the therapeutic agent is administered topically in a pharmaceutically acceptable excipient.

In another aspect the present invention concerns a method and a pharmaceutical composition wherein the pharmaceutical composition is administered topically to retard or stop cancers of the skin.

In another aspect the present invention concerns a method and a pharmaceutical composition wherein the pharmaceutical composition is administered intranasally or as part of inhalation therapy to retard or stop cancers of the lung.

In another aspect the present invention concerns a method and a pharmaceutical composition wherein the pharmaceutical composition is administered intravenously to retard or stop cancers of the breast, prostate, liver, kidney or any other internal organs or tissues.

In another aspect the present invention concerns a method and a pharmaceutical composition wherein the pharmaceutical composition is administered is a vaginal suppository to retard or stop cancers of the cervix, uterus or ovary.

In another aspect the present invention concerns a method and a pharmaceutical composition wherein the pharmaceutical composition is administered as an anally applied suppository to retard or stop cancers of the lower gastro-intestinal tract.

In another aspect the present invention concerns a method and a pharmaceutical composition wherein the pharmaceutical composition is administered orally to retard or stop cancers of the colon, upper gastrointestinal tract, breast, prostate, liver, kidney or any other internal organs or tissues.

In another aspect the present invention concerns a method and a pharmaceutical composition wherein the pharmaceutical composition is administered intramuscularly or subcutaneously as a general protection against cancer development in internal organs.

In another aspect, the present invention concerns a method of targeting and binding non-acetylated H3, H4 histones and other histone -variants such as the centromere-specific H3 variant, CENP-A, which method comprises:

A. Prevention of acetylation of amino acid residues found in N-terminal tail of H3, H4 and variant histones,

B. Prevention of phosphorylation of amino acid residues found in N-terminal tails of H3, H4 and variant histones.

C. Prevention of methylation of amino acid residues found in N-terminal tails of H3, H4 and variant histones.

D. Prevention of other post-translational modifications of amino acid residues found in N-terminal tails of H3, H4 and variant histones, with the result.

In another aspect, the present invention concerns a composition of matter, that is required to allow targeting and binding of proteins to non-acetylated H3, H4 histones and other histone —variants such as the centromere-specified H3 variant, CENP-A, which composition comprises:

A. Presence of a helical motif that is structurally conserved, comprising a consensus sequence of 9 amino acid residues, composed of: (ED)NNXXXEK(IV), where E is glutarnic acid, D is aspartic acid, I is isoleucine, V is valine, K is lysine residues, N is any amino acid, and X is conserved hydrophobic residues, and the

B. Presence of a block of 5-10 residues of acidic amino acids (either E is glutamic acid or D is aspartic acid), upstream or downstream of the helical motif.

### BRIEF DESCRIPTION OF THE FIGURES

FIGS. 1A, 1B, 1C, 1D and 1E are schematic representations oflunasin found in storage parenchyma cells and co-localizes with endoreduplicated DNA.

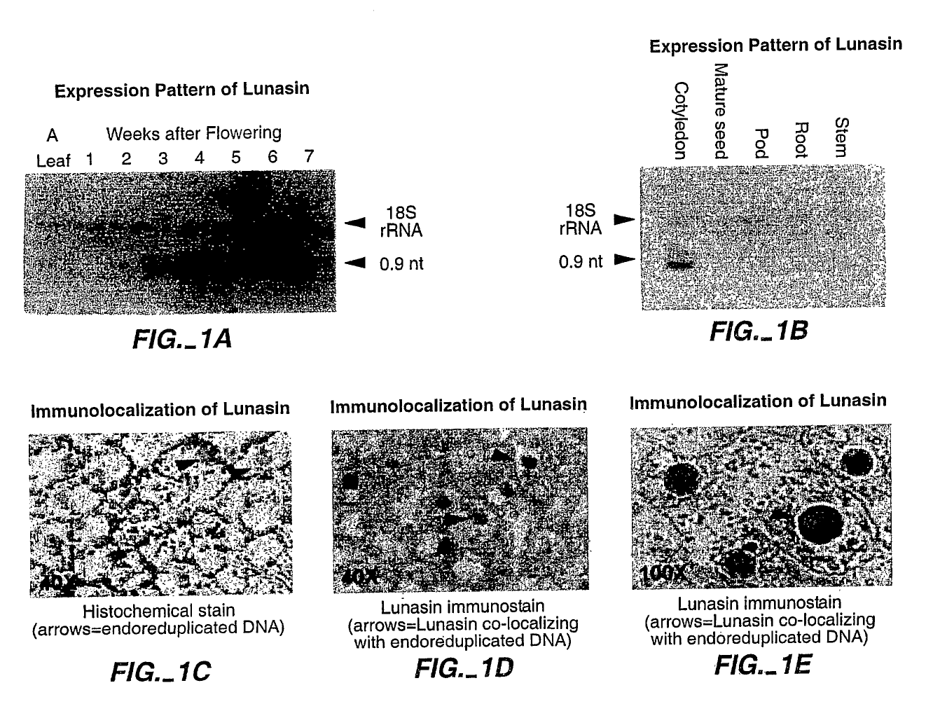

FIG. 2 is a graphic representation of relative cell adhesion versus amount of peptide added for lunasin and lunasin (-GRC) as it attaches to mammalian cell membrane through its RGD motif.

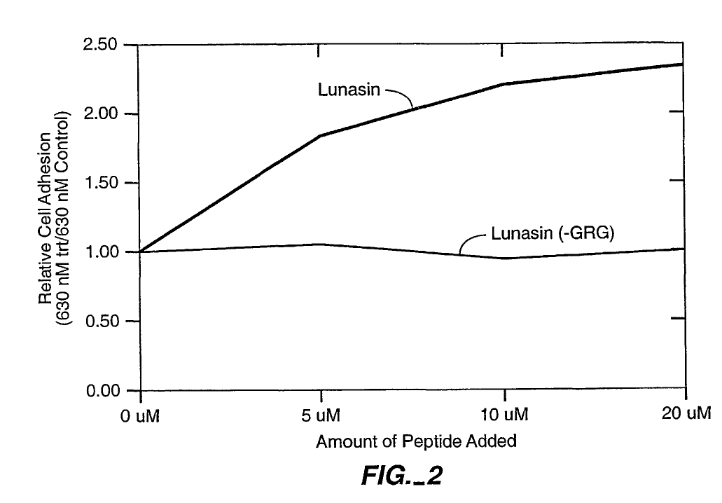

FIGS. 3A, 3B, 3C, 3D, 3E and 3F are schematic representations of lunasin adhering to the cell membrane and then becoming internalized.

FIGS. 4A and 4B are schematic representations of lunasin as a major constituent of the Bowman Birk protease inhibitor (BBIC) preparation.

FIG. 5 is a graphic representation of how lunasin inhibits carcinogen-induced transformation.

FIG. 6 is a graphic representation of lunasin in prevention of carcinogen-induced tumorous foci formation in normal cells.

FIGS. 7A, 7B, 7C, 7D, 7E and 7F are photographic representations of C3H cells transfected with E1A-Δ CR1, in the absence of lunasin and the presence of lunasin which induces apoptosis.

FIG. 8 is a schematic representation and model for the prevention of cancer in the presence of lunasin.

FIG. 9 is a graphic representation showing lunasin preferentially binding to deacylated histone H4.

FIG. 10 is a graphic representation showing the dose response oflunasin, trLunasin-del and NLS-trLunasin to increasing amounts of deacetylated H4 peptide.

FIG. 11 is a table which compares motifs showing that lunasin contains a helical motif having high structural homology to other chromatin binding proteins.

FIG. 12 is a graphic representation of the effect of modified lunasin peptides on transformation assay.

FIG. 13 is a schematic representation depicting how lunasin binds to deacylated histones and inhibits histone acylation.

### DETAILED DESCRIPTION OF THE INVENTION AND PREFERRED EMBODIMENTS

Definitions

As used herein:

The standard literature definitions found in articles and reference books are to be used to determine the definitions of the terms as found herein.

“Amino acid” refers to any of the naturally occurring amino acids having standard designations, G, V, K, I, W, etc. It also refers to those known synthetic amino acids.

Conserved hydrophobic amino acid refers to but are not limited to, for example, histidine, isoleucine, valine, methionine, alanine, or tyrosine.

“Lunasin” refers to compounds comprising the natural and recombinantly produced soybean lunasin polypeptide (coincidentally purified and sequenced by Odani et al., 1987 (Ser-Lys-Trp-Gln-His-Gln-Gln-Asp-Ser-Cys-Arg-Lys-Gln-Leu-Gln-Gly-Val-Asn-Leu-Thr-Pro-Cys-Glu-Lys-His-Ile-Met-Glu-Lys-Ile-Gln-Gly-Arg-Gly-Asp -Asp-Asp-Asp-Asp-Asp-Asp-Asp-Asp (SEQ. ID. 1).

“Lunasin” refers to the biologically active lunasin peptide having 1-43 amino acids.

“Lunasin or an active variant thereof” refers to the biologically active lunasin peptide having 43 amino acids, or to portions of the 1-43 amino acid chain which are also biologically active (shown herein as 22-43 amino acids meaning amino acid 22 to amino acid 43 of lunasin). See sequence data below:

protein having amino acids 1 to 42 (SEQ. ID. 2),

protein having amino acids 1 to 41 (SEQ. ID. 3),

protein having amino acids 1 to 40 (SEQ. ID. 4),

protein having amino acids 1 to 39 (SEQ. ID. 5),

protein having amino acids 1 to 38 (SEQ. ID. 6).

protein having amino acids 22 to 43 (SEQ. ID. 7),

protein having amino acids 22 to 42 (SEQ. ID. 8),

protein having amino acids 22 to 41 (SEQ. ID. 9),

protein having amino acids 22 to 40 (SEQ. ID. 10),

protein having amino acids 22 to 39 (SEQ. ID. 11), and

protein having amino acids 22 to 38 (SEQ. ID. 12)

Combinations of these active protein are also included.

Polyacidic amino acids refer, for example, to glutamic acid or aspartic acid.

1. The Lunasin Peptide has Anti-carcinogenic Property

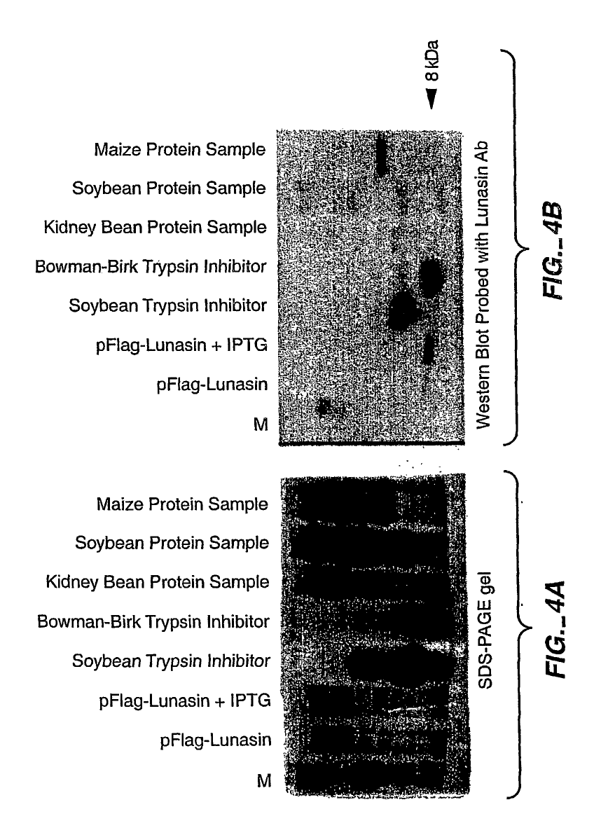

Lunasin has been shown to be a major constituent of the Bowman Birk protease inhibitor (BBIC) preparation (FIGS. 4A and 4B). BBIC has been shown to be chemopreventive in several in vitro and animal model studies (Examples: Yavelow et al., 1985 PNAS, vol. 82:5395; Weed et al., 1985 Carcinogenesis, vol 6:1239; Messadi et al., 1986 JNCI, vol. 76:447; Baturay, et al., 1986 Cell Biol and Toxic, vol. 2:21; St. Clair et. al., 1990 Cancer Res, vol 50:580; Reviews: Kennedy et al., 1993 Preventive Med, vol 22:796; Kennedy et al., 1995 J Nutr, vol. 125:733S). The evidence for the anti-carcinogenic effect of BBIC was compelling enough that NCI is now conducting human clinical trials (currently in Phase II) to prove its effectivity (Kennedy et al., 1993 Preventive Med, vol. 22:796). However, despite the accumulated in vitro and in vivo data pointing to the anticarcinogenic property of BBIC, the underlying mechanism of action has not been elucidated. More importantly, several scientific evidence have shown that BBIC or protease inhibitors (PI), in general, are unlikely to be the active anticarcinogenic component found in soybean. For one, cooked soy products, which are devoid of any protease inhibitor activity, are equally as effective at reducing cancer development as raw soy products (Clawson, 1996 Cancer Invest., vol. 14(6):608). The effect of protease inhibitors appears to be indirect because dietary PI are, in general, poorly absorbed from the gastro-intestinal (GI) tract, and never reach target organs in any measurable quantity (Clawson, 1996).

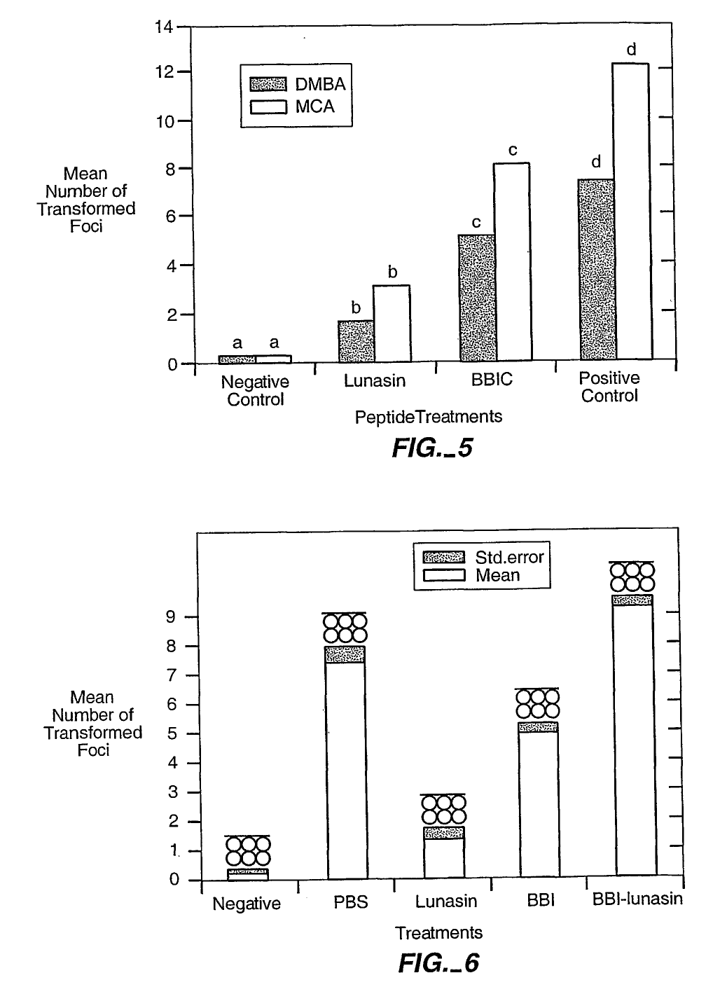

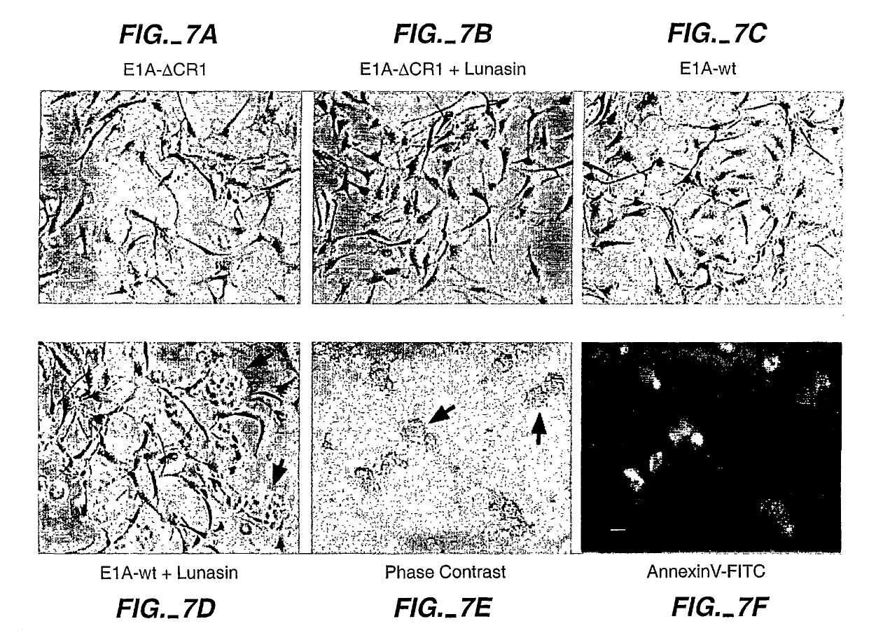

Lunasin is responsible for the cancer preventive activity attributed to BBIC, specially since the lunasin peptide is a significant contaminant in the BBIC preparation. Cell transformation assays conducted at UC Berkeley showed that lunasin was on average twice more effective than equimolar amounts (125 nM) of BBIC in reducing foci formation in C3H 10 T½ cells treated with potent chemical carcinogens, 7, 12-dimethylbenz[a]anthracene (DMBA) and 3-methylcholanthrene (CA) (FIG. 5). More importantly, BBIC with immunodepleted lunasin, prepared by applying commercially available BBI (Sigma T9777) through cationic exchange and immuno-affinity columns and then collecting flow through fractions, showed significant loss of its anti-transformation property (FIG. 6). The duplicated sets of experiments showed that BBIC with immunodepleted lunasin did not inhibit foci formation upon carcinogen treatment, similar to the effect of the untreated positive control. These results indicate that lunasin is the major cancer preventive ingredient in the BBIC preparation.

What then is the role of BBI in the cancer preventive property attributed to the BBIC soybean preparation? As pointed out by Clawson (1996), the effect of BBI appears to be indirect. Digestion experiments have shown that lunasin by itself gets broken down by pancreatic digestive enzymes but resists digestion when a chymotrypsin inhibitor like BBI is mixed with lunasin at equimolar ratios (Pascual and de Lumen, personal communication). It is most likely that BBI's role is to prevent the digestion of lunasin in the gut to allow intact lunasin to be absorbed through the gastro-intestinal tract. Once in the circulatory system, lunasin can be distributed to the various tissues and can get inside somatic cells by attaching to specific integrin receptors found in cell membranes through its RGD cell adhesion motif. Inside the cell, lunasin then preferentially binds to regions of the chromosomes enriched with hypoacetylated chromatin upon nuclear membrane breakdown at prometaphase.

2. Anti-carcinogenic Property of Lunasin: A Molecular Model Based on Lunasin Binding to Deacetylated Histones and Inhibition of Histone Acetylation.

The affinity of the lunasin peptide to regions of hypoacetylated chromatin suggests that lunasin may be involved in chromatin modification. Regulation of the post-translational modification of chromatin has been implicated in cel-cycle control and in how tumor suppressors act as critical downstream effectors during carcinogenesis (R. A. DePinho. Nature, vol. 391, 533 (1998)). Lunasin also contains a functional cell adhesion motif, Arg-Gly-Asp (RGD), which allows exogenously applied lunasin to bind and become internalized in mammalian cells. The presence of the RGD motif and its chromatin-binding characteristic point to a potential anti-carcinogenic role for lunasin.

Histone acetylation is associated with transcriptional activity in eukaryotic cells, having been observed mainly in transcriptionally active chromatin (K. Struhl, Genes Dev., vol. 12, 599 (1998); M. Grunstein, Nature, vol. 389,349 (1997)). The inhibition of histone acetylation by lunasin provides a mechanistic model to explain the anti-carcinogenesis property of this soybean peptide. The Rb tumor suppressor, a critical downstream effector during carcinogenesis (R. A. Weinberg, Cell, vol. 81, 323 (1995); M. C. Paggi, et al., J Cell. Biochem., vol. 62, 418 (1996)), was hypothesized to repress a subset of E2F-regulated genes by binding to the E2F family of DNA-binding transcription factors and by recruiting a histone deacetylase (HDAC1) to maintain a hypoacetylated state of condensed chromatin around the transcription start site (A. Brehrn et al., Nature, vol. 391, 597 (1998); L. Managhi-Jaulin et al. Nature, vol. 391, 601 (1998); R. X. Luo, A. A. Postigo, D. C. Dean, Cell, vol. 92, 463 (1998)). This dual repression mechanism is abrogated upon Rb inactivation during carcinogenesis, resulting in the release of Rb binding to the E2F promoter, acetylation of the repressed chromatin structure and the induction of expression of the E2F-regulated genes involved in cell proliferation (A. Brehm et al., Nature, vol. 391, 597 (1998); L. Managhi-Jaulin et al. Nature, vol. 391, 601 (1998); R. X. Luo, A. A. Postigo, D. C. Dean, Cell, vol. 92, 463 (1998)).

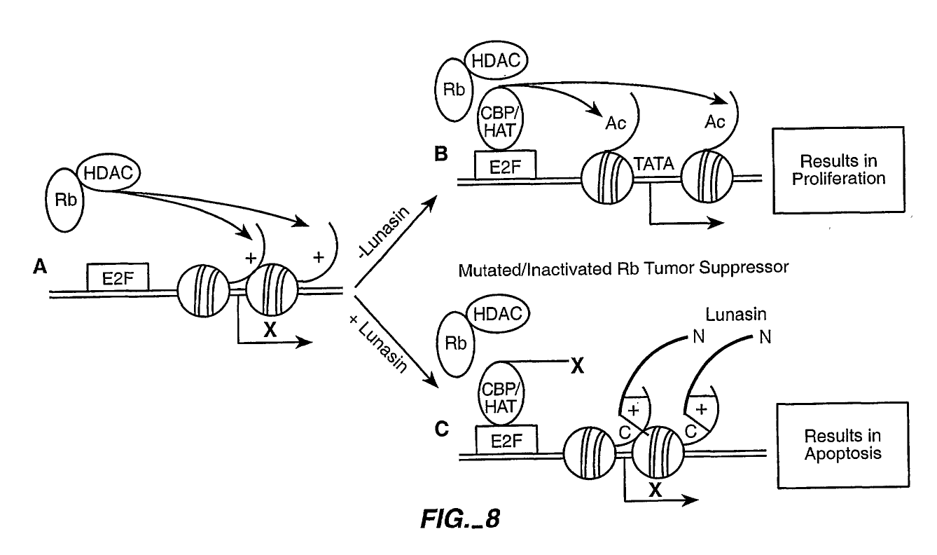

By binding to deacetylated histones found in repressed chromatin, it was hypothesized that lunasin can prevent cell proliferation and transformation even in the absence of a functional Rb by inhibiting histone acetylation and activation ofE2F-regulated genes. To test this molecular model of lunasin action, C3H cells were first treated with lunasin and then transfected with E1 A viral oncogene that specifically induces cell proliferation by binding and inactivating Rb (J. R. Nevins, Science, vol. 258, 424 (1992)). As a negative control, E1A with deleted conserved region 1 (E1AΔCR1) that abolishes the RB binding domain was likewise used in the transfection experiments (D. Trouche, T. Kouzidares, Proc. Natl. Acad. Sci., USA, vol. 93, 1439 (1996)). C3H cells transfected with E1A-ΔCR1, as expected, showed normally dividing cells at 20 h after transfection, both in the presence and absence of lunasin (FIGS. 7A, 7B, 7C, 7D, 7E and 7F). Transfection with the E1Awt in the absence of lunasin also showed normal cell proliferation (FIGS. 7A, 7B, 7C, 7D, 7E and 7F). However, C3H cells initially treated with lunasin for 24 h and then transfected with E1Awt resulted in the preponderance of non-adherent cells in solution at 20 h after transfection. Phase contrast image of the non-adherent cells showed characteristic morphology of apoptotic cells which was confirmed by the positive fluorescent staining for Annexin V-HITC (FIGS. 7A, 7B, 7C, 7D, 7E and 7F.).

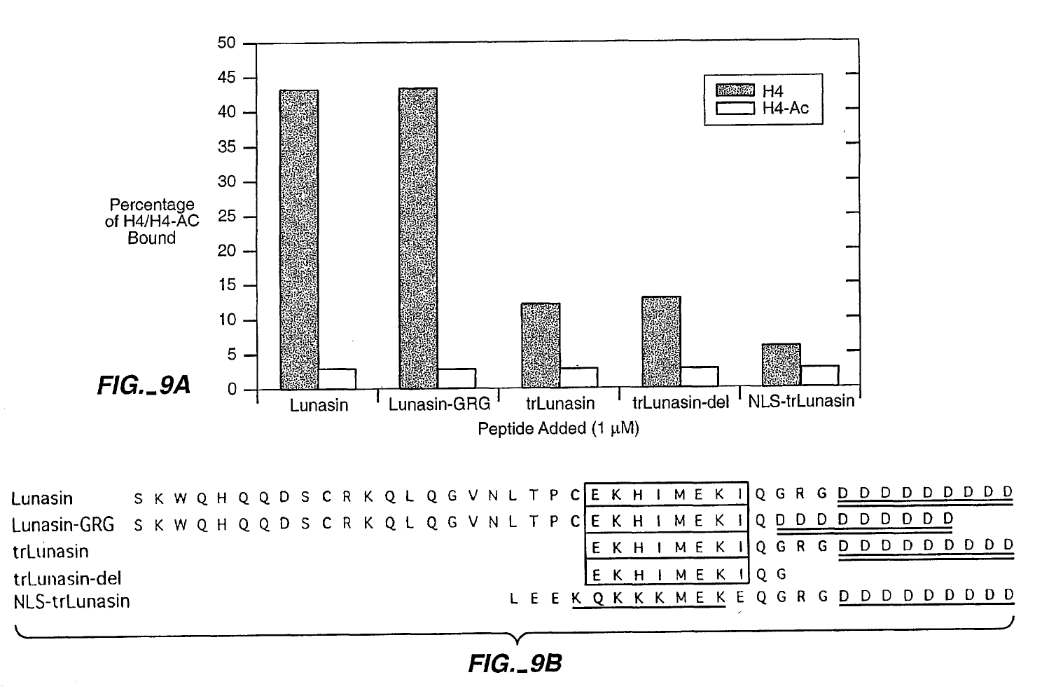

The induction of apoptosis by lunasin in E1A-transfected C3H cells provides evidence to a mechanistic model explaining lunasin's suppression of carcinogen-mediated transformation (FIG. 8). The Rb tumor suppressor inhibits the expression of E2F-regulated genes in part by tethering a histone deacetylase (HDAC1) to maintain a condensed hypoacetylated chromatin around the transcription start site (A. Brehm et al.,Nature, vol. 391, 597 (1998); L. Managhi-Jaulin et al. Nature, vol. 391, 601 (1998); R. X. Luo, A. A. Postigo, D. C. Dean, Cell, vol. 92, 463 (1998)). The inactivation of Rb by carcinogen treatment and oncogene expression results in the loosening up of the repressed chromatin structure by localized histone acetylation (R. H. Giles, D. J. Peters, M. H. Breuning, Trends Genet., vol. 14, 178 (1998)). This consequently results in the activation of genes involved in cell proliferation, which eventually leads to carcinogenesis. When lunasin is present in normal cells before Rb is inactivated, the deacetylated N-terminal tails of histone H3 and H4 found in repressed chromatin presumably bind to the acidic carboxyl end of lunasin. This makes these deacetylated histones unavailable as substrates for histone acetylation, thus maintaining the repressed chromatin structure around the E2F promoter even when carcinogens and the viral oncogene, E1A, inactivate Rb. The inhibition of expression of E2F-regulated genes triggers apoptosis instead of cell proliferation, which normally occurs when these genes are activated during carcinogenesis. The induction of apoptosis in cells with inactivated Rb by the presence of lunasin can explain the reduced number of transformed foci in normal murine fibroblast cells that have been treated with potent chemical carcinogens.

UTILITY AND ADMINISTRATION—Administration of the compounds of this invention can be via any of the accepted modes of administration for therapeutic agents. These methods include oral, parenteral, transdermal, subcutaneous and other modes.

Depending on the intended mode, the composition may be in many forms, for example, solid, semi-solid, or liquid dosage forms, including tablets, time release agents, pills, capsules, suspensions, solutions and the like. The compositions will include a conventional pharmaceutical excipient and an active compound as described herein or the pharmaceutically acceptable salts thereof and may, in addition, include other medicinal agents, pharmaceutical agents, carriers, adjuvants, diluents, etc.

The amount of the active compound administered will, of course, be dependent on the molecular weight of selected compound, the subject being treated, the subject's weight, the severity of the affliction, the manner of the administration and the judgment of the prescribing physician. However, an effective dose is in the range of about 0.1-500 mg/kg/day, preferably about 1-200 mg/kg/day. For an average 70 kg human, those dosages would amount to between about 0.01 to 35 g/day.

For solid compositions, conventional nontoxic solids include for example, pharmaceutical grades of manitol, lactose, starch, magnesium stearate, cellulose and the like may be used. Liquid pharmaceutically administratable compositions can be prepared by dissolving, dispersing, etc., a compound and optional pharmaceutical adjuvants in an excipient, such as, for example, water, glycerol, ethanol, vegetable oil and the like to form a suspension.

Actual methods of preparing such dosage forms are known, or will be apparent to those skilled in the art; see, for example, Remington's Pharmaceutical Sciences, Mack Publishing Company, Easton, Pa., 15^{th} Edition, 1975.

For instance for topical or intranasal or intravenous administration, the minimum dose is about 250 microg lunasin per mL of solution or per gram of solid dose up to a maximum dose of about 2.5 millig lunasin per mL of solution or per gram of solid dose.

The following preparations and examples serve to illustrate the invention. They should not be construed as narrowing it, nor as limiting its scope.

Experimental—General

The starting materials described herein are available from commercial supply houses, from recognized contracting organizations or can be prepared from published literature sources. Unless otherwise noted the material solvents, reagents, etc. are used as received without modification.

### EMBODIMENTS OF THE INVENTION

The experimental evidence described above point to the utility of the lunasin peptide in disrupting specific cellular processes like carcinogenesis. The proposed lunasin mechanism of action involves its preferential binding to the deacetylated N-terminal tails of histone H3 and H4, making them unavailable as substrates for acetylation. Since the acetylation of histone H3 and H4 is associated with gene activation, lunasin acts as a repressor of gene expression when it binds to deacetylated histones found in promoter regions of negatively regulated genes ( such as the family of E2F-regulated genes that are negatively regulated by the Rb tumor suppressor). The ability of lunasin to repress gene expression by preferential binding to deacetylated histones and preventing their acetylation has practical wide-ranging biological and therapeutic applications.

The invention describes the identification of the functional motif in the lunasin peptide responsible for its chromatin-binding property and its ability to inhibit acetylation of H3 and H4 histones. This invention is important for designing future drugs involving targeted repression of genes and for practical application in biological research by providing a method to target modified lunasin peptides to specific genes or genome locations and for the study of phenotypic effects of gene inactivation and silencing.

### EXAMPLE 1

### BINDING OF LUNASIN AND FRAGMENTS THEREOF

(a) The lunasin peptide preferentially binds to deacetylated histones and is mediated by a helical region in the carboxyl end.

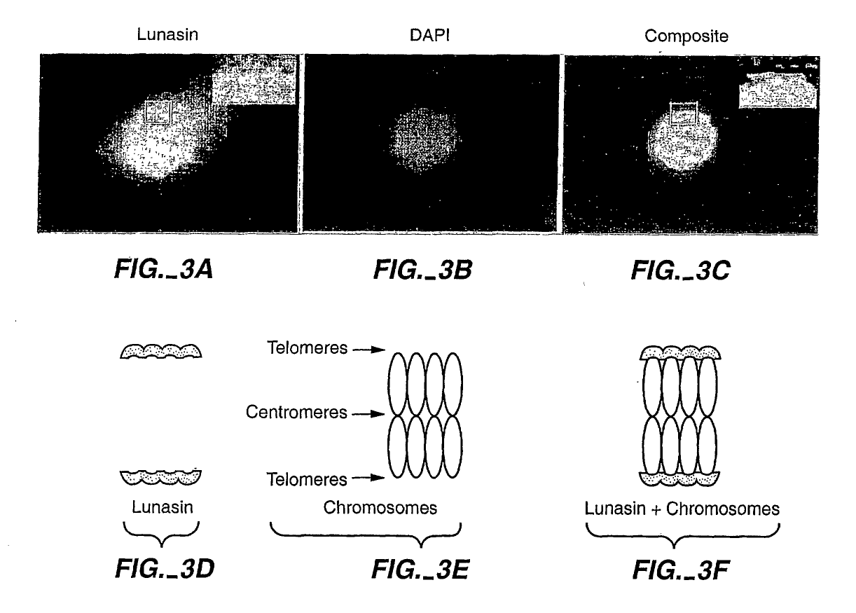

The antimitotic effect of the lunasin gene in transfected mammalian cells has been attributed to the competitive binding of lunasin to centromeres as visualized by GFP fluorescence and immunostaining (Galvez and de Lumen, 1999). On the other hand, immunostaining of exogenously applied lunasin revealed the preferential binding of lunasin mainly to the telomeres of metaphase chromosomes (FIG. 3). Telomeres, like the centromeres are genomic regions that are also rich in hypoacetylated chromatin, comprising mainly of deacetylated histones (Braunstein et al., Genes Dev, vol. 7, 592,1993). The increased affinity of lunasin to these regions may be due to the greater electrostatic attraction of the negatively charged carboxyl end of lunasin to the positively charged N-terminal tails of deacetylated histones.

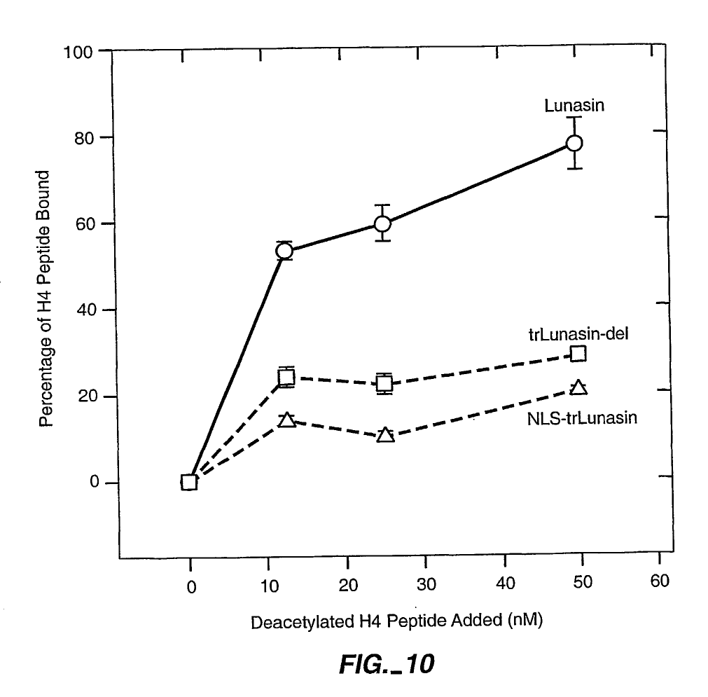

To test whether lunasin binds preferentially to deacetylated histones, an in vitro immuno-binding assay was conducted using acetylated and deacetylated forms of the H4 N-terminal tail (assay protocol was described in Galvez and de Lumen, 1999). The full lunasin peptide (Lunasin) and lunasin with deleted RGD motif (Lunasin-GRG) were found to bind with high affinity to deacetylated H4 N-terminus but not to the tetra-acetylated H4 (FIG. 9). This suggests that lunasin binds with high specificity to deacetylated H4 and that the RGD-motif is not important to its binding affinity. However, there was a significant reduction in deacetylated H4 binding for truncated lunasin (trLunasin) that contains only the reactive carboxyl end of the peptide. This indicates that the N-terminus of lunasin is also important for binding to deacetylated histones most likely by stabilizing the lunasin structure to allow electrostatic interactions between the carboxyl end of lunasin and deacetylated H4 to occur at higher efficiency.

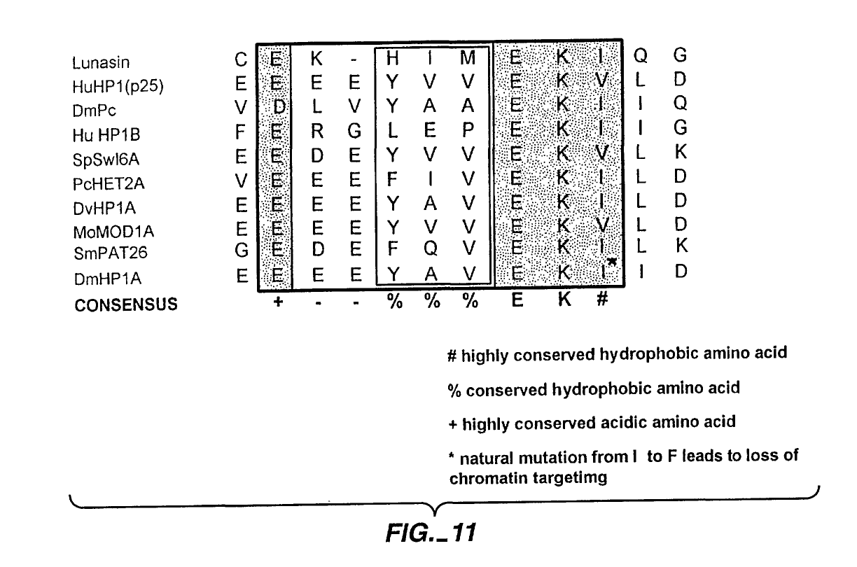

A comparison of the binding affinity of lunasin, tr-Lunasin and NLS-trLunasin to increasing dose of deacetylated H4 peptide showed an increase in lunasin binding when the amount of deacetylated H4 peptide added in the immuno-binding reaction is increased (FIG. 10). Lunasin binding to deacetylated H4 was 3×more than trLunasin-del, which in return was found to bind at significantly higher affinity than the NLS-trLunasin (FIG. 10).

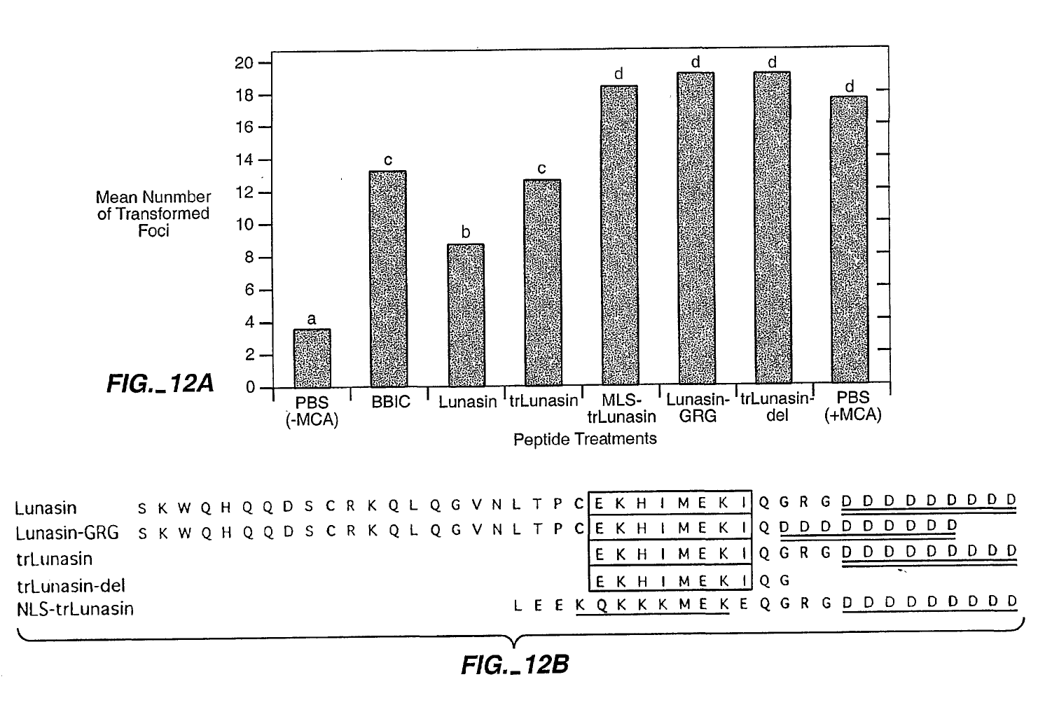

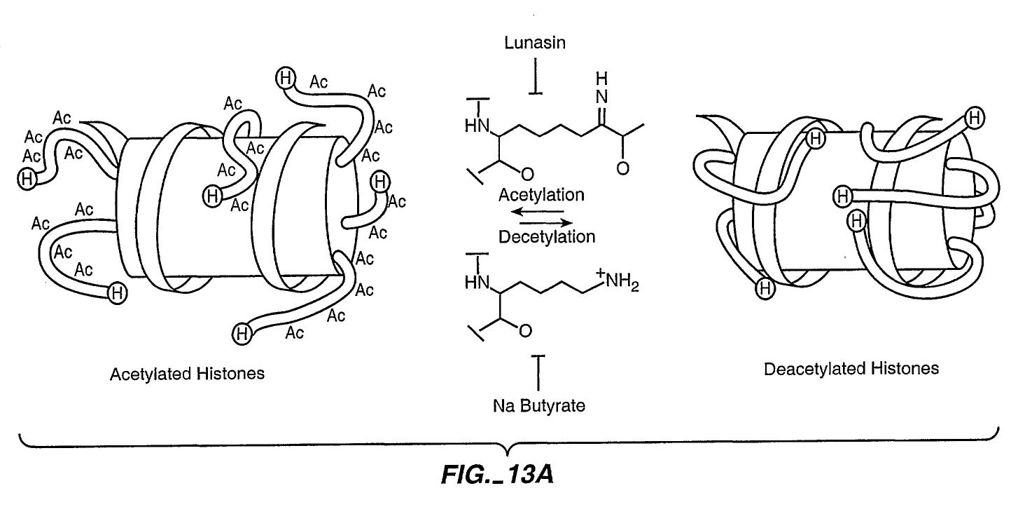

The binding affinity of trLunasin to deacetylated H4 was not significantly different from that of the 10 amino acid trLunasin-del peptide fragment. The trLunasin-del fragment spans a helical domain (B. Rost, C. Sander, Proteins, vol. 19, 55 (1994); B. Rost, C. Sander, J Mol. Biol., vol 232, 584 (1993) ) upstream of the poly-aspartyl carboxyl end of the lunasin peptide. The substitution of this helix by a nuclear localization sequence (NLS) in the truncated lunasin peptide (NLS-trLunasin) resulted in the loss of binding to deacetylated H4 (FIG. 9). This indicates that this helical region of lunasin may play a role in the binding of lunasin to deacetylated histones. A homology search of this helical region revealed structural similarity to a short, conserved region of the chromo-domain structure (R. Aasland, A. F. Stewart, Nucleic Acids Res, vol.. 23, 3168 (1995)) found in chromatin-binding proteins such as Drosophila and human heterochromatin (DmHP1A and HuHP1B, respectively) (FIG. 11). A naturally occurring mutation in Drosophila DmHP1A that converts isoleucine to phenylalanine (I to F mutation) (FIG. 11) led to the disruption of the helical motif and the consequent loss of chromatin targeting (S. Messmer, A. Franke, R. Paro, Genes Dev., vol. 6, 1241 (1992)). The presence of this helical motif in lunasin could explain the specific targeting of the peptide to deacetylated chromatin. Its absence from the NLS-trLunasin peptide reduced the binding to deacetylated H4 significantly, despite the presence of the poly-aspartyl end (FIG. 9). However, the presence of both helix and poly-aspartyl end was necessary for binding to deacetylated H4 (FIG. 9) and for the anti-transformation property of the truncated lunasin (trLunasin) peptide (FIG. 11). The poly-aspartyl end attached to this helical motif at the carboxyl end appears to be important for the anti-carcinogenic property of lunasin. Although the helical motif is necessary for targeting the lunasin peptide to deacetylated histones, it is the acidic poly-aspartyl end that interacts with the positively charged non-acetylated lysine residues in the histone N-terminal tails preventing them from being acetylated. It should also be pointed out that trLunasin has a lower binding affinity to deacetylated H4 than the full-length lunasin peptide (FIG. 9). This observation correlates with the reduced efficacy of trLunasin in preventing foci transformation (FIG. 12). This result provides evidence linking the binding affinity of lunasin to deacetylated histones and its anti-transformation property in vivo preferably in a human being.

(b) Similarly when the reaction involving lunasin (SEQ.ID. 1 of 43 amino acids) of step (a) is repeated except that the lunasin is replaced by a stoichiometrically equivalent and active fragment selected from:

protein having amino acids 1 to 42 (SEQ. ID. 2),

protein having amino acids 1 to 41 (SEQ. ID. 3),

protein having amino acids 1 to 40 (SEQ. ID. 4),

protein having amino acids 1 to 39 (SEQ. ID. 5),

protein having amino acids 1 to 38 (SEQ. ID. 6).

protein having amino acids 22 to 43 (SEQ. ID. 7),

protein having amino acids 22 to 42 (SEQ. ID. 8),

protein having amino acids 22 to 41 (SEQ. ID. 9),

protein having amino acids 22 to 40 (SEQ. ID. 10),

protein having amino acids 22 to 39 (SEQ. ID. 11), and

protein having amino acids 22 to 38 (SEQ. ID. 12),

a corresponding useful therapeutic result is obtained in cancer inhibition and in reduction of cancer activity in vivo.

### EXAMPLE 2

### INHIBITION OF IN VIVO ACETYLATION

(a) Lunasin binding to deactylated histones inhibits in vivo acetylation of histone H3 and H4

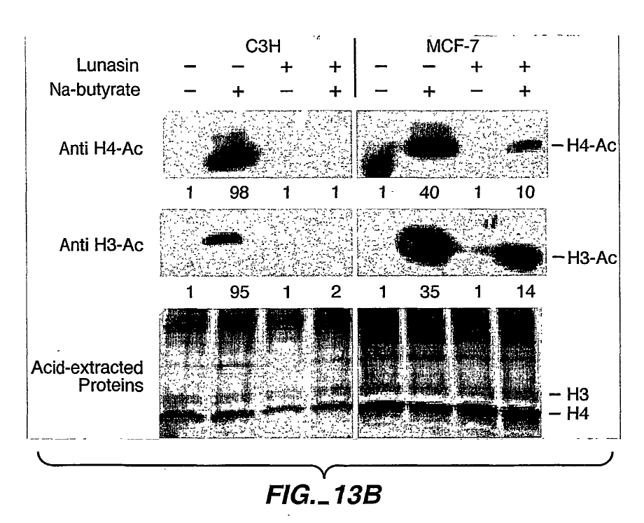

The in vitro binding of lunasin to deacetylated histone H4 confirms the observed affinity of lunasin to regions of hypoacetylated chromatin such as the centromeres and telomeres in immunostaining experiments (Galvez and de Lumen, 1999 and FIG. 6). Deacetylated histones are substrates for histone acetylation and for chromatin remodelling which has been associated with eukaryotic transcriptional regulatory mechanisms (K Struhl, Genes Dev., vol. 12, 599,1998; M. Grunstein, Nature, vol. 389,349, 1997). To determine whether the preferential binding of lunasin to deacetylated histones has any biochemical effect on histone acetylation in vivo, C3H cells and the human breast cancer cell line, MCF-7, were treated with the histone deacetylase inhibitor, Na-butyrate (E. P. Candido, R. Reeves, J. R. Davie, Cell, vol. 14, 105,1978), in the presence or absence of lunasin. Immunoblots of acid-extracted proteins show the significant reduction of acetylated H4 and H3 in Na-butyrate treated C3H and MCF-7 cells when pretreated with 1 μM of lunasin peptide (FIG. 13). The absence of lunasin when cells were treated with Na-butyrate increased histone H4 acetylation by 200 fold in both C3H and MCF-7 cells. H3 acetylation induced by Na-butyrate treatment increased 100 fold in C3H cells and around 400 fold in MCF-7 cells. Upon addition of lunasin, there was no observed increase in H4 and H3 acetylation of C3H cells treated with Na-butyrate. In MCF-7 cells, H4 acetylation was reduced 10 fold and H3 acetylation 4 fold when lunasin was added prior to Na-butyrate treatment. These results demonstrate that the exogenous application of the lunasin peptide inhibit histone acetylation of mammalian cells in vivo, preferably in a human being.

(b) Similarly when the reaction involving lunasin (SEQ.ID. 1 of 43 amino acids) of step (a) is repeated except that the lunasin is replaced by a stocchiometrically equivalent and active fragment selected from:

protein having amino acids 1 to 42 (SEQ. ID. 2),

protein having amino acids 1 to 41 (SEQ. ID. 3),

protein having amino acids 1 to 40 (SEQ. ID. 4),

protein having amino acids 1 to 39 (SEQ. ID. 5),

protein having amino acids 1 to 38 (SEQ. ID. 6).

protein having amino acids 22 to 43 (SEQ. ID. 7),

protein having amino acids 22 to 42 (SEQ. ID. 8),

protein having amino acids 22 to 41 (SEQ. ID. 9),

protein having amino acids 22 to 40 (SEQ. ID. 10),

protein having amino acids 22 to 39 (SEQ. ID. 11), and

protein having amino acids 22 to 38 (SEQ. ID. 12),

a corresponding useful therapeutic result is obtained in cancer inhibition and in reduction of existing cancer activity in vivo, preferably in a human being.

While only a few general embodiments of the invention have been shown and described herein, it will become apparent to those skilled in the art that various modifications and changes can be made in the application of lunasin and lunasin analogs and active lunasin fragments thereof to treat existing tumors or prevent initiation of tumor formation without departing from the spirit and scope of the present invention. All such modifications and changes coming within the scope of the appended claims are intended to be carried out thereby.

I claim

1. A method of cancer treatment or prevention, which method comprises:
A. Administering to a mammalian subject having tumor cells in need of therapy or a mammalian subject at risk to carcinogen or oncogene-mediated cancer formation an effective amount of an isolated and purified therapeutic agent selected from the group consisting of lunasin peptide, an active fragment of lunasin peptide, an active lunasin peptide analog and combinations thereof which lunasin moiety has a helical portion which the structural motif (ED)NNXXXEK(IV), where E is glutamic acid, D is aspartic acid, K is lysine, I is isoleucine, V is valine, X is selected from conserved hydrophobic amino acids and N is any amino acid, a sequence of at least 5 to about 15 poly-acidic amino acids selected from glutamic acid or aspartic acid, and an Arg-Gly-Asp (RGD) motif which is useful for targeting and binding to non-acetylated N-terminal tails of H4 and H3 histones and for functional adhesion of lunasin moiety to the outer cell membrane;
B. Causing the lunasin peptide, the active fragment of lunasin peptide, the active lunasin peptide analog or combinations thereof to contact and to adhere to the functional cell membrane;
C. Causing the lunasin peptide, the active fragment of lunasin peptide, the active lunasin peptide analog or combinations thereof to become internalized within the functioning cell;
D. Causing the lunasin peptide, the active fragment of lunasin peptide, the active lunasin peptide analog or combinations thereof to preferentially bind to the deacylated N-terminal portions of histone H3 and H4, causing these histones to be unavailable for further acylation in regions of the chromosomes of the cell and which are enriched with hypoacylated repressed chromatin;
E. Inducing apoptosis of the cell by repression of carcinogen-mediated gene transformation within the cell, which results in significantly reduced or termination of cancer activity of existing tumor cells or the prevention of significant tumor cell initiation.

2. The method of claim 1 wherein the mammal is a human being.

3. The method of claim 1 wherein the method is one of treating an already existing cancer.

4. The method of claim 1 wherein the method is one of preventing or repressing the induction of cancer.

5. The method of claim 1 wherein the therapeutic agent comprises lunasin peptide.

6. The method of claim 1 wherein the therapeutic agent comprises an active fragment of lunasin peptide.

7. The method of claim 6 wherein the active fragment of lunasin is selected from the group consisting of:
protein having amino acids 1 to 42 (SEQ. ID. 2),
protein having amino acids 1 to 41 (SEQ. ID. 3),
protein having amino acids 1 to 40 (SEQ. ID. 4),
protein having amino acids 1 to 39 (SEQ. ID. 5),
protein having amino acids 1 to 38 (SEQ. ID. 6).
protein having amino acids 22 to 43 (SEQ. ID. 7),
protein having amino acids 22 to 42 (SEQ. ID. 8),
protein having amino acids 22 to 41 (SEQ. ID. 9),
protein having amino acids 22 to 40 (SEQ. ID. 10),
protein having amino acids 22 to 39 (SEQ. ID. 11),
protein having amino acids 22 to 38 (SEQ. ID. 12), and combinations thereof

8. The method of claim 1 wherein the therapeutic agent comprises an active analog of lunasin peptide.

9. The method of claim 1 wherein the therapeutic dose is about 250 microgram per milliliter or per gram of solid dose to about 2.5 milligram per milliliter or per gram of solid dose.

10. The method of claim 1 wherein the therapeutic agent is administered orally, topically, intranasally, intramuscularly, subcutaneously, intraperioneally, buccally intravenously or combinations of these methods.

11. The method of claim 1 wherein the therapeutic agent is administered topically in a pharmaceutically acceptable excipient.

12. The method of claim 1 wherein the therapeutic agent is administered orally.

13. A pharmaceutical composition which comprises a lunisin peptide, an active fragment of lunasin peptide, an active lunasin peptide analog or combinations thereof and a pharmaceutically acceptable excipient.

14. The pharmaceutical composition of claim 13 which comprises a lunisin peptide and a pharmaceutically acceptable excipient.

15. The pharmaceutical composition of claim 13 which comprises an active fragment of lunasin peptide, and a pharmaceutically acceptable excipient.

16. The pharmaceutical composition of claim 13 wherein the active fragment of lunasin peptide is selected from the group consisting of:
protein having amino acids 1 to 42 (SEQ. ID. 2),
protein having amino acids 1 to 41 (SEQ. ID. 3),
protein having amino acids 1 to 40 (SEQ. ID. 4),
protein having amino acids 1 to 39 (SEQ. ID. 5),
protein having amino acids 1 to 38 (SEQ. ID. 6).
protein having amino acids 22 to 43 (SEQ. ID. 7),
protein having amino acids 22 to 42 (SEQ. ID. 8),
protein having amino acids 22 to 41 (SEQ. ID. 9),
protein having amino acids 22 to 40 (SEQ. ID. 10),
protein having amino acids 22 to 39 (SEQ. ID. 11),
protein having amino acids 22 to 38 (SEQ. ID. 12), and combinations thereof.

17. The pharmaceutical composition of claim 13 which comprises an active lunasin peptide analog and a pharmaceutically acceptable excipient.

18. The pharmaceutical composition of claim 13 wherein the therapeutic dose is about 250 microg per milliliter or per gram of solid dose to about 2.5 millig per milliliter or per gram of solid dose.

19. The pharmaceutical composition of claim 13 wherein said pharmaceutical composition is administered orally, topically, intranasally, intramuscularly, subcutaneously, intrapertineally, buccally or combinations of these methods.

20. The pharmaceutical composition of claim 13 wherein said pharmaceutical composition is administered topically to retard or stop cancers of the skin.

21. The pharmaceutical composition of claim 13 wherein said pharmaceutical composition is administered intranasally or as part of inhalation therapy to retard or stop cancers of the lung.

22. The pharmaceutical composition of claim 13 wherein said pharmaceutical composition is administered intravenously to retard or stop cancers of the breast, prostate, liver, kidney or any other internal organs or tissues.

23. The pharmaceutical composition of claim 13 wherein said pharmaceutical composition is administered is a vaginal suppository to retard or stop cancers of the cervix, uterus or ovary.

24. The pharmaceutical composition of claim 13 wherein said pharmaceutical composition is administered as an anally applied suppository to retard or stop cancers of the lower gastrointestinal tract.

25. The pharmaceutical composition of claim 13 wherein said pharmaceutical composition is administered orally to retard or stop cancers of the colon, upper gastrointestinal tract, breast, prostate, liver, kidney or any other internal organs or tissues.

26. The pharmaceutical composition of claim 13 wherein said pharmaceutical composition is administered intramuscularly or subcutaneously as a general protection against cancer development in internal organs.

27. The pharmaceutical composition of any of claims 19 to 26 wherein the active fragment of lunasin peptide is selected from the group consisting of:
protein having amino acids 1 to 42 (SEQ. ID. 2),
protein having amino acids 1 to 41 (SEQ. ID. 3),
protein having amino acids 1 to 40 (SEQ. ID. 4),
protein having amino acids 1 to 39 (SEQ. ID. 5),
protein having amino acids 1 to 38 (SEQ. ID. 6).
protein having amino acids 22 to 43 (SEQ. ID. 7),
protein having amino acids 22 to 42 (SEQ. ID. 8),
protein having amino acids 22 to 41 (SEQ. ID. 9),
protein having amino acids 22 to 40 (SEQ. ID. 10),
protein having amino acids 22 to 39 (SEQ. ID. 11), and
protein having amino acids 22 to 38 (SEQ. ID. 12),

28. The pharmaceutical composition of any of claims 19 to 26 wherein the therapeutic dose is about 250 microgram per milliliter or per gram of solid dose to about 2.5 milligram per milliliter or per gram of solid dose.
